(19) United States
(12) Patent Application Publication
(10) Pub. No.: US 2017/0350884 A1
(43) Pub. Date: Dec. 7, 2017

(54) RHEUMATOID ARTHRITIS RELATED BIOMARKER

(71) Applicant: The Catholic University of Korea Industry-Academic Cooperation Foundation, Seoul (KR)

(72) Inventors: Wan-Uk Kim, Seoul (KR); Daehee Hwang, Gyeongsangbuk-do (KR); Yune-Jung Park, Gyeonggi-do (KR); Sung Yong You, Gyeongsangbuk-do (KR); Seung-Ah Yoo, Gyeonggi-do (KR)

(21) Appl. No.: 15/521,133

(22) PCT Filed: Oct. 22, 2015
(86) PCT No.: PCT/KR2015/011228

§ 371 Date: Apr. 21, 2017

(30) Foreign Application Priority Data
Oct. 24, 2014 (KR) 10-2014-0145062

(51) Int. Cl. G01N 33/564

(52) U.S. Cl. CPC G01N 33/564; C12Y 304/16001; G01N 2333/948; G01N 2800/56; G01N 2800/102

(57) ABSTRACT

The present invention relates to a biomarker for diagnosing rheumatoid arthritis, a biomarker for diagnosing osteoporosis as a complication of rheumatoid arthritis, a biomarker for predicting the severity and prognosis of rheumatoid arthritis, and a use thereof.

### TECHNICAL FIELD

The present invention relates to a biomarker for diagnosing rheumatoid arthritis, a biomarker for diagnosing osteoporosis as a complication of rheumatoid arthritis, a biomarker for predicting severity and prognosis of rheumatoid arthritis, and a use thereof.

### BACKGROUND ART

Rheumatoid arthritis (RA) is a disease caused by inflammation of a tissue, such as synovium, that surrounds joints, which is a typical chronic disease in which about 1% of the total population in Korea is estimated to be a patient.

Although the diagnosis of rheumatoid arthritis depends primarily on clinical symptoms, there is a limit that diagnosis is made after the destruction of the joint has progressed considerably, and a rheumatoid factor (RF) as a serologic marker of rheumatoid arthritis is included in the diagnostic criteria of the American College of Rheumatology (ACR), but there is a problem that the RF shows to be negative through process of disease in 20% of rheumatoid arthritis patients, and the RF appears in chronic inflammations, malignant tumors, and even some healthy elderly people, so that there is a disadvantage that it has a low specificity.

In addition, as a method of treating rheumatoid arthritis, a drug treatment method is used in which an analgesic anti-inflammatory agent is generally administrated in combination with various antirheumatic agents in order to minimize joint damage, prevent loss of function and reduce pain, and recently, biological therapeutic agents have been developed and is being used in combination therapy with antirheumatic medicines, and when the severity of the disease is serious, an operative therapy is being performed. However, even though such a treatment method has an excellent effect, since there are disadvantages that the joint deformation may continue, some cases may have difficulty to suitably treat the disease due to drug side effects and the treatment costs a large amount due to the drug price increase according to development costs of new drugs, it is urgent to develop methods capable of suitably diagnosing and predicting onset, prognosis and severity of rheumatoid arthritis.

Furthermore, it has been reported that in rheumatoid arthritis patients, two types of osteopenia, that is, periarticular osteopenia and systemic osteoporosis, are observed and they have the increased risk of fracture compared to the general population (Sambrook P N, et al., Determinants of axial bone loss in Rheumatoid arthritis, Arthritis Rheum 1987, 30: 721-8, Compston J E, et al., Spinal trabecular bone mineral content in patients with non-steroid treated rheumatoid arthritis Ann Rheum Dis 1988, 47: 660-4). The systemic osteoporosis has overall osteopenia in axial skeleton or appendicular skeleton, like osteoporosis occurring in metabolic bone diseases. To date, it is noted that the pathogenesis and the cause of osteoporosis in rheumatoid arthritis patients are controversial and various factors complexly act on. The risk factors for osteoporosis in rheumatoid arthritis patients known until now include age, sex, presence or absence of menopause, obesity, use of adrenal corticosteroids, disease activity, long duration of disease, etc. (Gough A K S et al. With rheumatoid arthritis. Lancet 1994, 344: 23-7). Osteoporosis ultimately increases the risk of fracture, and to reduce social and economical losses due to the fractures, it is very important to find high-risk groups at an early stage, and prevent and actively treat them. In particular, it can be seen as urgent to find biomarkers that can detect the risk of osteoporosis early in patients with high risk factors of osteoporosis such as rheumatoid arthritis.

### DISCLOSURE

### Technical Problem

It is an object of the present invention to provide a composition or kit for diagnosing rheumatoid arthritis, comprising an agent for measuring the level of expression of cathepsin A, and a method for diagnosing rheumatoid arthritis using the same.

It is also an object of the present invention to provide a composition or kit for diagnosing osteoporosis in a rheumatoid arthritis patient, comprising an agent for measuring the level of expression of one or more selected from the group consisting of cathepsin A, sCD14 (soluble CD14) and AGP1 (alpha-1-acid glycoprotein 1), and a method for diagnosing osteoporosis in a rheumatoid arthritis patient using the same.

It is also an object of the present invention to provide a composition or kit for predicting severity and prognosis of rheumatoid arthritis, comprising an agent for measuring the level of expression of AGP2 (alpha-1-acid glycoprotein 2), and a method for predicting severity and prognosis of rheumatoid arthritis using the same.

### Technical Solution

In one aspect for achieving the above object, the present invention relates to a composition for diagnosing rheumatoid arthritis, comprising an agent for measuring the level of expression of cathepsin A.

In another aspect, the present invention relates to a kit for diagnosing rheumatoid arthritis comprising the composition.

In another aspect, the present invention relates to a method for detecting cathepsin A from a urine sample of a patient to provide information necessary for diagnosis of rheumatoid arthritis.

In another aspect, the present invention relates to a composition for diagnosing osteoporosis in a rheumatoid arthritis patient, comprising an agent for measuring the level of expression of one or more selected from the group consisting of cathepsin A, sCD14 (soluble CD14) and AGP1 (alpha-1-acid glycoprotein 1).

In another aspect, the present invention relates to a kit for diagnosing osteoporosis in a rheumatoid arthritis patient, comprising the composition.

In another aspect, the present invention relates to a method for detecting at least one selected from the group consisting of cathepsin A, sCD14 and AGP1 from a urine sample of a patient to provide information necessary for diagnosis of osteoporosis in a rheumatoid arthritis patient.

In another aspect, the present invention relates to a composition for predicting severity and prognosis of rheumatoid arthritis, comprising an agent for measuring the level of expression of AGP2 (alpha-1-acid glycoprotein 2).

In another aspect, the present invention relates to a kit for predicting severity and prognosis of rheumatoid arthritis, comprising the composition.

In another aspect, the present invention relates to a method for detecting AGP2 from a urine sample of a patient to provide information necessary for diagnosis of rheumatoid arthritis.

### Advantageous Effects

The biomarkers provided by the present invention can be widely utilized in predicting onset of rheumatoid arthritis, onset of complications, and severity and prognosis, and appropriate treatments complying with the patient's condition can be performed.

### BRIEF DESCRIPTION OF DRAWINGS

FIG. 1A shows the results comparing urinary cathepsin A levels in rheumatoid arthritis patients (RA) and a control group (Control). The control group includes patients with degenerative osteoarthritis (OA) and patients with systemic lupus erythematosus (SLE) (**P<0.01). FIG. 1B shows the results comparing urinary cathepsin A levels in rheumatoid arthritis patients (RA), patients with degenerative osteoarthritis (OA) and patients with systemic lupus erythematosus (SLE) (**P<0.01).

FIG. 2 shows the results comparing urinary AGP1 levels in rheumatoid arthritis patients according to bone mineral density extents [Normal, Osteopenia, and Osteoporosis] in femurs of the rheumatoid arthritis patients. FIG. 2A shows the results at the femur necks, where Normal vs. Osteopenia vs. Osteoporosis appeared in 0.311±0.016 vs. 0.318±0.022 vs. 0.387±0.031 ng/ml, FIG. 2B shows the results at the femur trochanters, where Normal vs. Osteopenia vs. Osteoporosis appeared in 0.305±0.014 vs. 0.336±0.025 vs. 0.397±0.040 ng/ml, FIG. 2C shows the results at the femur intertrochanters, where Normal vs. Osteopenia vs. Osteoporosis appeared in 0.312±0.013 vs. 0.313±0.028 vs. 0.404±0.028 ng/ml, and FIG. 2D shows the results under the average bone mineral density of the femur upper triangles (Femur total), where Normal vs. Osteopenia vs. Osteoporosis appeared in 0.306±0.014 vs. 0.320+0.024 vs. 0.395±0.029 ng/ml. **P<0.05

FIG. 3 shows the results comparing urinary sCD14 levels in rheumatoid arthritis patients according to bone mineral density extents [Normal, Osteopenia, and Osteoporosis] in femurs of the rheumatoid arthritis patients. FIG. 3A shows the results at the femur necks, where Normal vs. Osteopenia vs. Osteoporosis appeared in 158.2±13.0 vs. 167.4±27.3 vs. 261.7±69.7 ng/ml, FIG. 3B shows the results at the femur trochanters, where Normal vs. Osteopenia vs. Osteoporosis appeared in 147.1±12.0 vs. 223.8±36.2 vs. 237.7±55.8 ng/ml, FIG. 3C shows the results at the femur intertrochanters, where Normal vs. Osteopenia vs. Osteoporosis appeared in 146.4±11.8 vs. 216.9±34.7 vs. 249.1±77.9 ng/ml, and FIG. 3D shows the results under the average bone mineral density of the femur upper triangles (Femur total), where Normal vs. Osteopenia vs. Osteoporosis appeared in 145.4±11.6 vs. 223.5±32.9 vs. 210.7±55.6 ng/ml. *P<0.05

FIG. 4 shows the results comparing urinary cathepsin A levels in rheumatoid arthritis patients according to bone mineral density extents [Normal, Osteopenia, and Osteoporosis] in femurs of the rheumatoid arthritis patients. FIG. 4A shows the results at the femur trochanter, where Normal vs. Osteopenia vs. Osteoporosis appeared in 0.394±0.10 vs. 0.497±0.08 vs. 1.250±0.42 ng/ml, FIG. 4B shows the results at the femur necks, where Normal vs. Osteopenia vs. Osteoporosis appeared in 0.377±0.05 vs. 0.438±0.09 vs. 0.724±0.40 ng/ml, FIG. 4C shows the results at the femur intertrochanters, where Normal vs. Osteopenia vs. Osteoporosis appeared in 0.437±0.01 vs. 0.529±0.13 vs. 0.946±0.53 ng/ml, and FIG. 4D shows the results under the average bone mineral density of the femur upper triangles (Femur total), where Normal vs. Osteopenia vs. Osteoporosis appeared in 0.406±0.09 vs. 0.470±0.08 vs. 1.059±0.40 ng/ml. *P<0.05

FIG. 5 shows the results comparing urinary AGP2 protein levels in a patient group with rheumatoid arthritis that bone destruction processed (Radiographic progression) and a patient group without progression (Radiographic non-progression).

FIG. 6 shows the results comparing bone destruction prediction ability of CRP, ESR and AGP2 via ROC curve analyses in rheumatoid arthritis patients.

FIG. 7 shows the results identified with probability plots whether possibility of bone destruction prediction is improved when urine AGP2 is used in combination with blood CRP in rheumatoid arthritis patients.

### BEST MODE

Hereinafter, the present invention will be described in more detail.

In the present invention, the term “diagnosis” means identifying the presence or characteristic of a pathological condition. For the purpose of the present invention, the diagnosis may mean identifying the onset of rheumatoid arthritis, or further, whether or not the disease progresses or intensifies. In addition, the diagnosis herein may mean identifying the onset of osteoporosis, which is a typical complication in rheumatoid arthritis patients, or further, whether or not the disease progresses or intensifies.

In the present invention, the term “marker for diagnosis, marker for diagnosing or diagnosis marker” may mean a material that can diagnose rheumatoid arthritis by distinguishing it from states other than rheumatoid arthritis (e.g., by distinguishing it from normal, other arthritis such as osteoarthritis, or other autoimmune diseases). In addition, it may mean a material that can diagnose the onset state of osteoporosis, which is a typical complication in rheumatoid arthritis patients, by distinguishing it from the non-onset state. Such a material includes organic biomolecules, such as polypeptides or nucleic acids (for example, mRNA, etc.), lipids, glycolipids, glycoproteins or sugars (monosaccharides, disaccharides, oligosaccharides, etc.), which show increase or decrease in samples of subjects with diseases over samples of subjects that the disease is not developed. For purposes of the present invention, the diagnosis marker of the present invention may mean cathepsin A, which exhibits the specifically increased expression in a sample of a rheumatoid arthritis patient. For purposes of the present invention, the diagnosis marker of the present invention may also mean cathepsin A, AGP1 and/or sCD14 proteins which exhibit specifically increased expression in a sample of a patient with an occurrence of osteoporosis among rheumatoid arthritis patients.

In the present invention, the term “predicting severity and prognosis of rheumatoid arthritis” is related to possibility that bone destruction progresses or joint deformation is caused due to high disease activity when the overall degree of inflammation, degree of bone destruction, or degree of progression of rheumatoid arthritis has been evaluated in rheumatoid arthritis patients.

DAS (Disease Activity Score) as a representative method for measuring disease severity of rheumatoid arthritis in the conventionally clinical test or DAS28 (Disease Activity Score 28) as a modified form thereof is an index evaluation method of score computation and evaluates subjective factors evaluated by patients and physicians together with some objective factors such as inflammatory marker tests or radiographic findings. DAS28 is a composite index consisting of number of joints feeling tenderness of 28 joints in the patient, number of joints showing edema, erythrocyte sedimentation rate, and systemic evaluation of the patient, where the 28 joints include both shoulder joints, elbow and wrist joints, metacarpophalangeal joints, proximal interphalangeal joints, knee joints, and the like.

The disease activity of rheumatoid arthritis investigated by the DAS28 can be calculated from 0 to 9.4 points, and in general, if the DAS28 score is less than 2.6 points, it is defined as a state with little disease activity (remission), if the score is 2.6 points or more and less than 3.2 points, it is defined as low disease activity (minor), if the score is 3.2 points or more and less than 5.1 points, it is defined as moderate disease activity (moderate), and if the score is 5.1 points or more, it is defined as high disease activity (severe).

The present invention can easily predict the future disease severity of patients in a non-invasive method, which can be utilized as information for delay, alleviation and/or full recovery of the disease after the onset of rheumatoid arthritis by determining whether additional necessary treatment methods are used.

The present invention provides AGP2 as a prediction marker of severity and prognosis in rheumatoid arthritis patients. In the present invention, AGP2 is an index for capable of predicting the disease state of rheumatoid arthritis, which can predict that the risk of bone destruction in rheumatoid arthritis increases, when the concentration of AGP2 in the urine sample of the patient increases. It was confirmed that AGP2 did not only exhibit a power of test similar to C-reactive protein (CRP), which are an existing and well-known risk factor for predicting bone destruction, but also when the risk was calculated in combination of CRP and AGP2, a group which is CRP-positive and AGP2-positive increased the degree of bone destruction by 46.45 times compared with a group that both are negative. Therefore, it is possible to complementarily increase the prediction accuracy of rheumatoid arthritis severity not only when using AGP2 alone, but also when using it in combination with CRP.

In the present invention, “sCD14” is a soluble CD14, which is one of the forms of CD14. CD14 is a membrane surface protein of 55 kDa, linked to GPI (glycophosphatidylinositol), where for the amino acid sequence of sCD14, the sequence information thereof can be identified in a known gene database. For example, the amino acid sequence of human sCD14 protein can be identified in NCBI Genbank accession number NP_000582.1.

In the present invention, “AGP1 (a-1-acid glycoprotein 1)” and “AGP2 (a-1-acid glycoprotein 2)” are also referred to as orosomucoids 1 and 2, respectively, and are proteins which are derived in a stress state and secreted into plasma under stress conditions such as infection and inflammation. For the amino acid sequences of AGP1 and AGP2, the sequence information thereof can be identified in a known gene database. For example, the amino acid sequence of human AGP1 protein can be identified in NCBI Genbank accession number NP_000598.2, and the amino acid sequence of human AGP2 protein can be identified in NCBI Genbank accession number NP_000599.1.

In the present invention, for the amino acid sequence of cathepsin A, the sequence information thereof can be identified in a known gene database. For example, the amino acid sequence of human cathepsin A protein can be identified in NCBI Genbank accession number NP_000299.2.

In one example, an agent for measuring the level of expression of a marker herein may be an antibody specific to each marker.

In the present invention, “antibody” means a protein molecule specific to an antigenic site. For the purpose of the present invention, the antibody means an antibody that specifically binds to a marker such as cathepsin A, sCD14, AGP1 and/or AGP2, which is the marker protein, and comprises all of monoclonal antibodies, polyclonal antibodies, and recombinant antibodies.

The monoclonal antibody can be prepared using techniques of hybridoma methods (Kohler and Milstein (1976) European journal of Immunology 6: 511-519), or phage antibody libraries (Clarkson et al, Nature, 352: 624-628, 1991; Marks et al., J. Mol. Biol., 222: 58, 1-597, 1991) as widely known in the relevant field.

The polyclonal antibody can be produced by methods widely known in the relevant field that a serum comprising the antibody is obtained by injecting the above-described protein antigen into an animal and gathering blood from the animal. Such a polyclonal antibody can be prepared from any animal species host, such as goats, rabbits, sheep, monkeys, horses, pigs, cows, and dogs.

In addition, the antibody of the present invention also comprises special antibodies such as chimeric antibodies, humanized antibodies, and human antibodies.

Furthermore, the antibody used in the present invention comprises functional fragments of antibody molecules as well as complete forms with two full-length light chains and two full-length heavy chains. The functional fragment of the antibody molecule refers to a fragment having at least an antigen-binding function, which may exemplify Fab, F(ab′), F(ab′)2, Fv and the like.

As the method for measuring the level of expression of cathepsin A, sCD14, AGP1 and/or AGP2 in a sample using such an antibody, a method in which the degree of forming an antigen-antibody complex can be confirmed by processing the sample with the antibody may be used without limitation. The “antigen-antibody complex” means a binding substance of an antibody specific to a marker such as cathepsin A, sCD14, AGP1 and AGP2, and the formation amount of the antigen-antibody complex can be quantitatively measured via a signal size of a detection label.

For example, Western blot, an ELISA (enzyme linked immunosorbent assay), an immunoprecipitation assay, a complement fixation assay, a flow cytometry (Fluorescence Activated Cell Sorter, FACS), or a protein chip, and the like, may be exemplified, without being limited thereto.

In another example, the kit of the present invention may comprise one or more compositions, solutions, or devices suitable for assaying expression levels as well as an agent capable of measuring the level of expression of a marker in a patient sample. For example, the kit may comprise a substrate, a suitable buffer solution, a secondary antibody labeled with a detection label, and a chromogenic substrate for immunological detection of the antibody.

In a specific example, the kit may be a kit characterized by comprising essential elements necessary for performing an ELISA to implement various ELISA methods such as an ELISA kit and a sandwich ELISA. The ELISA kit comprises antibodies specific to the markers. The antibody is an antibody having high specificity and affinity for markers such as cathepsin A, sCD14, AGP1 and/or AGP2, and little cross reactivity with other proteins, which may be a monoclonal antibody, a polyclonal antibody or a recombinant antibody. The ELISA kit may also comprise antibodies specific to control proteins. Other ELISA kits may comprise reagents capable of detecting bound antibodies, for example, labeled secondary antibodies, chromopores, enzymes and other materials capable of binding to their substrates or antibodies, and the like.

In addition to this, the kit may be a kit for implementing Western blot, an immunoprecipitation assay, a complement fixation assay, a flow cytometry, or a protein chip, and the like and may further comprise additional configurations suitable for each assay method. Through these assay methods, it is possible to provide information for diagnosis or prognosis prediction related to rheumatoid arthritis by comparing the formation amount of the antigen-antibody complex, and accordingly to appropriately treat the patient.

In another example, the kit of the present invention may be provided in the form of a protein chip.

The protein chip is one in which at least one antibody for a marker such as cathepsin A, sCD14, AGP1 and/or AGP2 is arranged at a predetermined position and immobilized at a high density. The analysis method using the protein chip can confirm information for diagnosis or prognosis prediction related to rheumatoid arthritis by isolating a protein from a specimen sample, hybridizing the isolated protein with the protein chip to form an antigen-antibody complex and reading this to confirm the expression degree of the protein.

The formation amount of the antigen-antibody complex can be quantitatively measured via the signal size of the detection label.

The detection label may be selected from the group consisting of an enzyme, a fluorescent material, a ligand, a luminescent material, a microparticle, a redox molecule, and a radioisotope, but is not necessarily limited thereto.

In the present invention, the term “sample” includes a sample such as a tissue, a cell, whole blood, plasma, serum, blood, saliva, sputum, lymph, cerebrospinal fluid, intercellular fluid, or urine, which has different levels of expression of markers such as cathepsin A, sCD14, AGP1 and/or AGP2, but is not limited thereto. In a more preferred aspect, the sample may be a urine sample.

The process of isolating the protein from the specimen sample can be carried out using a known process, and the protein level can be measured by the above-mentioned various measurement methods of antigen-antibody complexes.

In a more preferred aspect, according to the present invention, the detection of the marker can be carried out by using an antibody specific to each marker.

In a more preferred aspect, the antibody may be a monoclonal antibody or a polyclonal antibody specific to each marker, but may further be a functional fragment of an antibody having antigen binding activity.

### MODE FOR INVENTION

Hereinafter, the present invention will be described in detail with reference to examples. However, the following examples are only illustrative of the present invention, and the present invention is not restricted by the following examples.

### Example 1. Experimental Material and Experimental Method

1-1. Selection of Subject Patients

Patients diagnosed with rheumatoid arthritis (RA) according to the American College of Rheumatology (ACR) criteria in 1987 were included as subjects. From patient medical records, age, sex, height, weight, disease duration and the like were each confirmed. Clinical factors, such as complete blood count (CBC), blood sugar, serum creatinine, serum albumin, erythrocyte sedimentation rate (ESR), C-reactive protein (CRP), rheumatoid factor (RF), and anti-cyclic citrullinated protein (CCP) antibodies which are specific antibodies for diagnosis of rheumatoid arthritis, of patients were evaluated. However, current smokers and patients with a history of cardiovascular diseases, uncontrolled hypertension patients (>160/100 mmHg), rheumatic vasculitis, amyloidosis, diabetic nephropathy, chronic renal disease patients (glomerular filtration rate <60 ml/min/1.732), current or chronic infection patients, thyroid or liver disease patients, and pregnant women with cancer were excluded from this study. Stable treatment for 3 months was also needed for this study. Patients who did not take angiotensin-converting enzyme inhibitors or angiotensin receptor blockers among antihypertensive drugs were included. X-rays of both hands and feet in the RA patients were also taken and these data were analyzed by specialists without knowing the status or condition of each patient. Bone erosion was measured and imaging severity was analyzed by the Sharp/van der Heijde method. The study protocol was approved by the Institutional Review Board of the Catholic Medical Center (XC09TIMI0070) and all experiments were progressed which received paper consents for this study protocol from all patients.

197 RA patients, and 75 degenerative osteoarthritis (OA) patients and 42 systemic lupus erythematosus (SLE) patients without kidney attack were recruited.

1-2. Protein Extraction from Urine Samples

Urine samples were collected at the time of daily routine diagnosis of patients. Albumin and creatine from the urine were measured using Hitachi7600-110 colorimetry. The collected urine samples were stored at −80° C. To remove unnecessary debris from the urine sample, centrifugation was carried out at 4° C. for 5 minutes at 2,000 g. Samples of degenerative osteoarthritis (OA) patients and systemic lupus erythematosus (SLE) patients were used as controls for rheumatoid arthritis. The urine sample was mixed with methanol at a ratio of 1:9 (v/v), incubated at −20° C. for 14 hours, and then centrifuged at 14,000 g at 4° C. for 30 minutes to extract proteins. The protein pellet was washed with methanol, dried in air and then resuspended in Tris buffer solution (5 mM EDTA, 50 mM Tris-HCl pH 7.5). The amount of the proteins was measured using Micro BCA Protein Assay Kit (Thermo Scientific, Rockford, Ill., USA).

1-3. Albumin Removal and Dialysis

To improve detection of urine proteins, albumin was removed using Affinity Removal Spin Cartridge, Human Albumin (Agilent Technologies, Wilmington, Del., USA). According to the manufacturer's protocol, the urine protein sample was diluted 3-fold with depletion buffer A (Agilent Technologies) and then filtered through a 0.22 μm spin filter (Agilent Technologies). The diluted urine sample was loaded in a spin cartridge and centrifuged at 100 g for 1.5 min. Flow-through fractions were collected and the cartridge was washed by centrifuging the sample with depletion buffer A at 100 g for 2.5 min. All flow-through fractions were mixed. The albumin-removed urine sample was dialyzed using Slide-A-Lyzer Dialysis Cassettes (Thermo Scientific, molecular weight cut-off of 3.5 kDa) to remove salts. To reduce the volume of the dialyzed sample, it was dried at high speed (speed-vac dry). The amount of the proteins was measured using Micro BCA Protein Assay Kit (Thermo Scientific, Rockford, Ill., USA).

1-4. ELISA (Enzyme-Linked Immunosorbent Assay)

To measure the levels of cathepsin A, sCD14, AGP1 and AGP2 in the urine, the ELISA was performed using an ELISA (USCN Life Science Inc., Missouri City, Tex., USA) or CD14 ELISA assay (R & D Systems, Minneapolis, Minn., USA).

1-5. Measurement of Bone Mineral Density

The bone mineral density was measured using the same model (GE lunar) in all patients. The lumbar spine bone mineral density was measured from Lumbar spine 1 (L1) to Lumbar spine 4 (L4), where the mean value of L2-L4 e was analyzed as the lumbar spine bone mineral density, and in the femur, the bone mineral density was measured at four sites of neck, trochanter, intertrochanter, and the average bone mineral density of the upper triangle (total). The bone mineral density results were analyzed using Z-score and T-score according to the purpose of analysis. Normal values of bone mineral density according to each age and sex were referred to the results of normal Korean controls. The Z-score was defined as (patient's measured BMD−normal BMD of age- and sex-matched controls)/standard deviation for age- and sex-matched controls, and the T-score was defined as (patient's measured BMD−maximal BMD of sex-matched controls)/standard deviation for maximal BMD of sex-matched controls.

1-6. Disease Severity Assessment

To investigate whether urinary proteins can predict the severity of disease (bone destruction degree) in rheumatoid arthritis patients, it was analyzed using the previously known bone destruction-related risk factors of rheumatoid arthritis, including anti-CCP antibodies (anti-cyclic citrullinated protein antibodies; anti-CCP Ab), erythrocyte sedimentation rate (ESR), C-reactive protein (CRP), and disease activity status (Lindqvist E, et al., 2002) Prognostic laboratory markers of joint damage in rheumatoid arthritis. Annals of the rheumatic diseases 2005, 64: 196-201).

The disease activity status was defined by “Disease Activity Score 28-joint assessment (DAS28) score.” DAS28 score <3.2 indicates low disease activity, 3.2≦DAS28 score <5.1 indicates moderate disease activity, and DAS28 score≧5.1 indicates high disease activity. The DAS28 score was calculated from the relationship including the erythrocyte sedimentation rate (ESR). Simple X-ray photographs of both hands and feet taken at the first outpatient visit of the patient and simple X-ray photographs of both his hands and feet taken after three years of follow-up observation were reviewed by two rheumatologists, where through the modified Sharp/van der Heijde (SvdH) method (van der Heijde D. How to read radiographs according to the Sharp/van der Heijde method. The Journal of rheumatology 2000, 27: 261-3) the bone erosion score was set to 1-4 points by scoring the number of erosions and it was set to 5 points when more than half of the joints were destroyed. Sixteen hand joints and six foot joints were evaluated with the bone erosion score of at most 5 points per each joint. Sixteen hand joints and six foot joints were also evaluated with 1 point for localized stenosis of joint spaces, 2 points for less than 50% stenosis of joint spaces, 3 points for 50% or more stenosis of joint spaces, and 4 points for joint stiffness. In the definition of imaging bone destruction progression, when the difference of simple X-ray total score of both hands and feet taken at the first outpatient visit from simple X-ray total score(bone erosion score+stenosis score of joint spaces) of both hands/feet taken after three years of follow-up observation is calculated and this difference is 4 points or more, it was defined as bone destruction progression (Giles J T, et al., Association of circulating adiponectin levels with progression of radiographic joint destruction in rheumatoid arthritis. Annals of the rheumatic diseases 2011, 70: 1562-8).

1-7. Statistical Analysis

The distribution for all variables was investigated. The variables representing normal distribution were expressed as mean±standard deviation (SD), and the variables representing non-normal distribution were indicated as median [interquartile range; IQR]. Comparisons among three patient groups were performed using Bonferroni correction and one-way analysis of variance (ANOVA) as a post hoc test. For categorical data, the difference in prevalence was assessed by chi-square test or Fisher's exact test. For cross-sectional comparisons between two groups, Mann-Whitney U test was used. For prediction of disease activity status and severity, ROC curves for urine AGP2, CRP, and ESR were compared.

All reported P values are a two-tailed test and a p value of 0.05 indicates statistical significance. When both side P values are <0.05, they were considered to be significant. They were analyzed using R software, version 2.14.1.

### Example 2. Comparison of Urinary Cathepsin a Levels in Rheumatoid Arthritis Patients and Controls

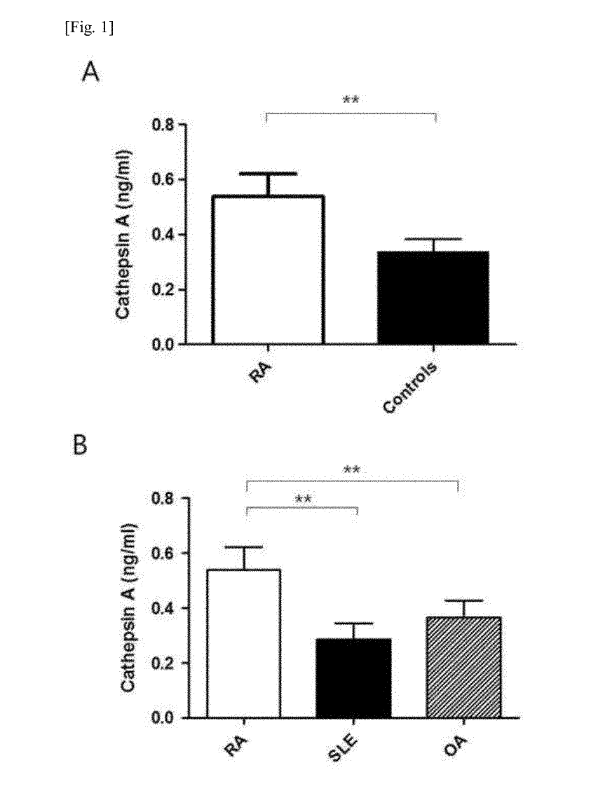

Levels of cathepsin A (human lysosomal protective protein) were measured by ELISA from 197 rheumatoid arthritis patients and 117 controls (75 degenerative osteoarthritis patients and 42 systemic lupus erythematosus patients without kidney attack). As a result, the urinary cathepsin A levels after urinary creatinine correction appeared as 0.287±0.058 in the systemic lupus erythematosus patients, 0.364±0.064 in the degenerative osteoarthritis patients, and 0.538±0.083 in the rheumatoid arthritis patients, so that the levels were significantly increased in rheumatoid arthritis patients compared to systemic lupus erythematosus patients and degenerative osteoarthritis patients (FIG. 1).

### Example 3: Search of Diagnosis Markers of Osteoporosis which is a Representative Complication of Rheumatoid Arthritis Patients

Patients, whose rheumatoid arthritis complies with the diagnostic criteria of the American College of Rheumatology (ACR), were included as subjects, but among them cases having thyroid diseases or otherwise occurring with internal diseases that may affect bone metabolism were excluded in the subjects. In addition, patients with higher bone mineral density (BMD) due to artificial compensating products and bone compression, such as patients who underwent compensating product implantation due to previous hip joint fractures and herniated disc, were excluded.

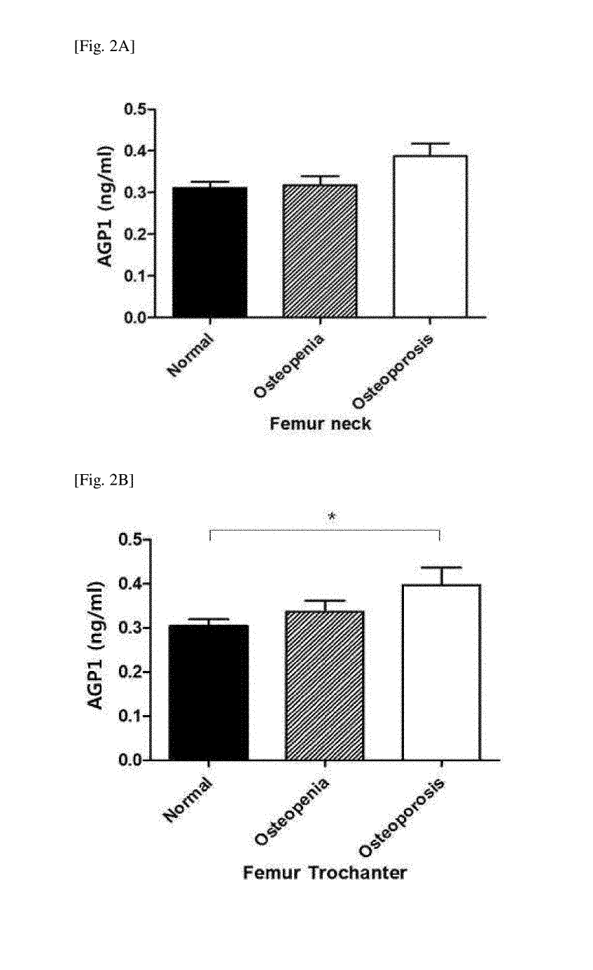

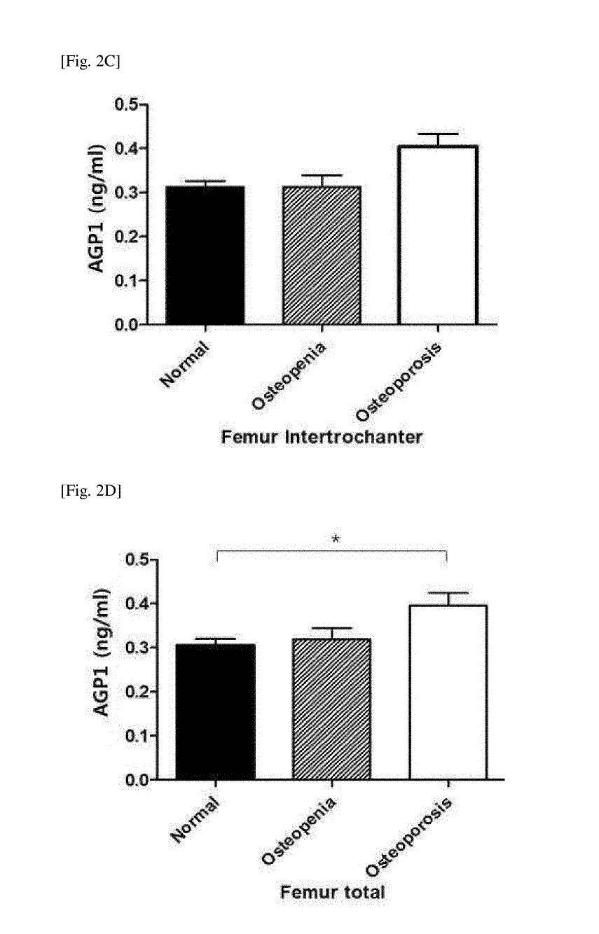

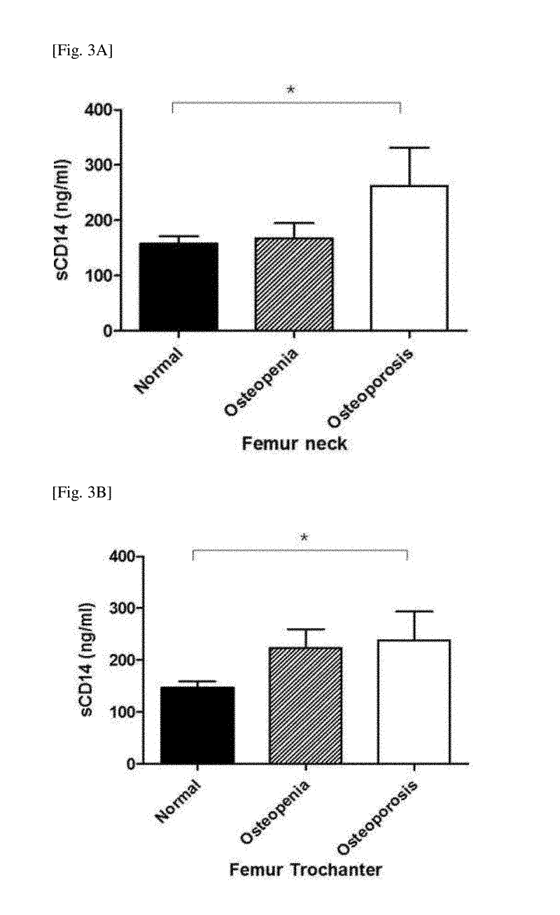

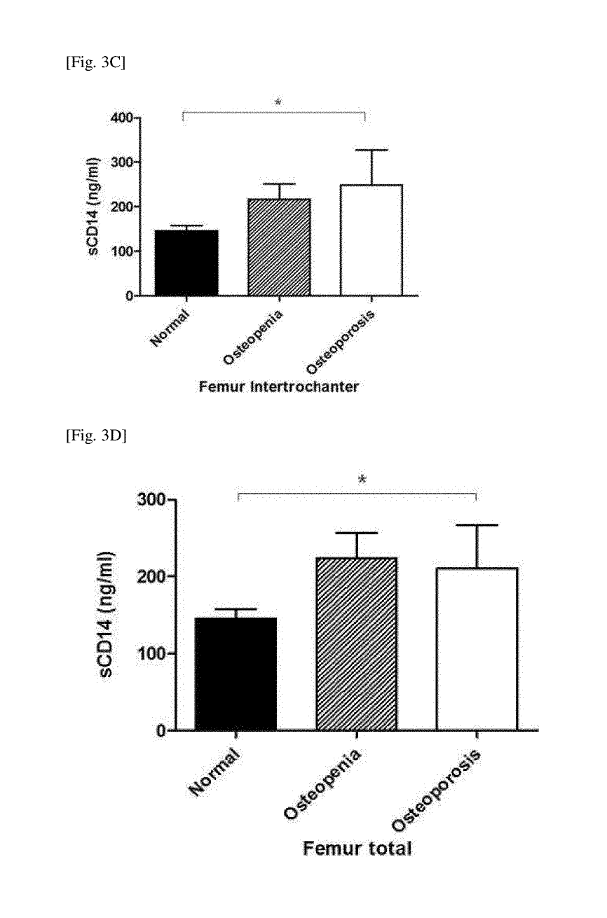

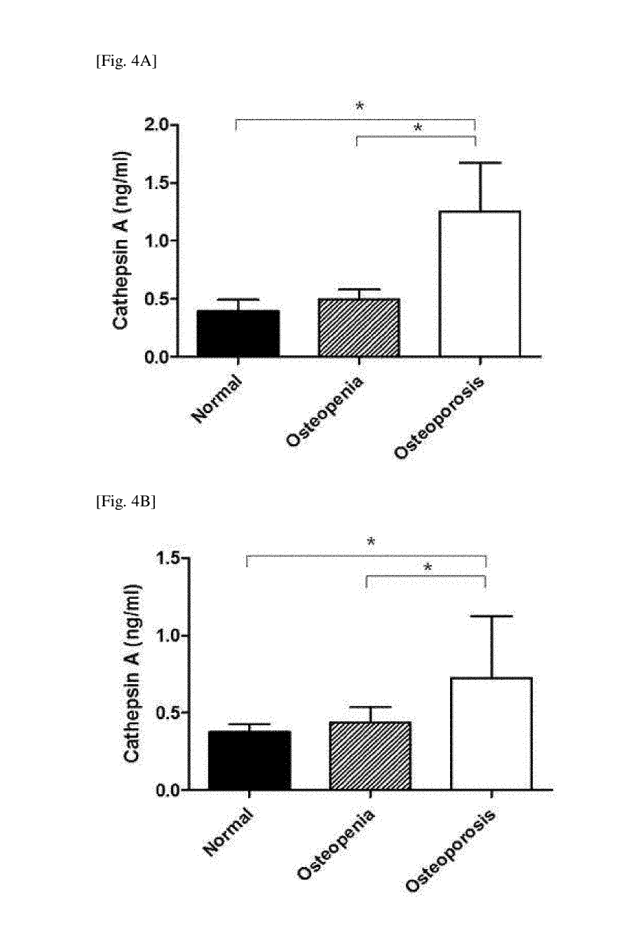

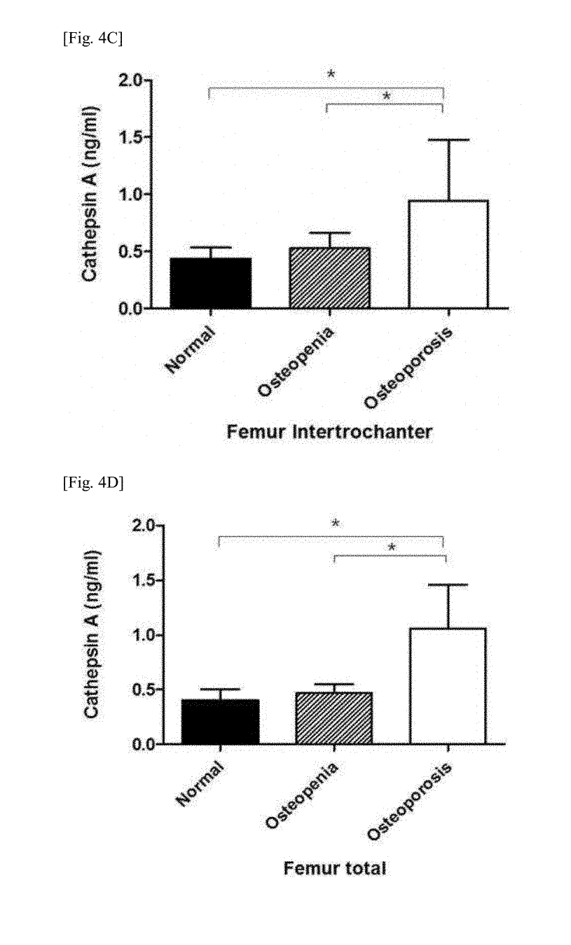

As a result of measuring the levels of AGP1, sCD14 and cathepsin A in the urine of the osteoporosis patients among the rheumatoid arthritis patients by the ELISA method, it was confirmed that the levels of AGP1 (FIG. 2), sCD14 (FIG. 3) and cathepsin A (FIG. 4) were significantly increased.

### Example 4. Search of Biomarkers Predicting Severity of Rheumatoid Arthritis Patients

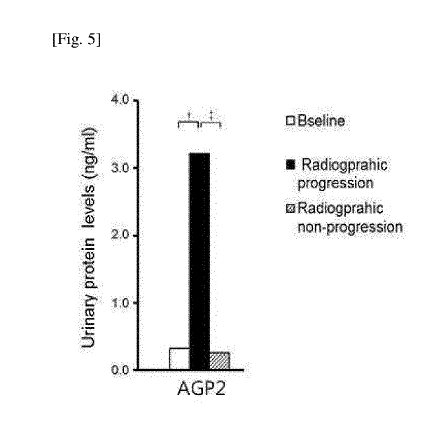

Three year follow-up observation was performed to investigate whether urinary proteins can predict disease severity (degree of bone destruction) in rheumatoid arthritis patients. The patients were divided into the group that bone destruction progressed (radiographic progression) and the group that it did not (radiographic non-progression), and the risk factors at the initial evaluation for each group were compared. As a result, in the group that bone destruction progressed, the AGP2 level was significantly increased compared to the initial evaluation (FIG. 5 and Table 1).

TABLE 1

| | | After three years | |
|---|---|---|---|
| | | Bone destruction | Bone destruction |
| | Baseline | progressed | non-progressed |
| (Unit: ng/ml) | (n = 282) | group (n = 24) | group (n = 184) |
| AGP2 | 0.333* | 3.216* | 0.261† |
| | (0.179-1.444) | (0.357-5.416) | (0.168-0.600) |

Data represent median and interquartile range.
*ANCOVA was used. P < 0.001
†Mann-Whitney U test was used. P < 0.001

In a univariate analysis of factors associated with progression of bone destruction, initial CRP level, ESR, and anti-CCP antibody status showed significance (Table 2).

TABLE 2

| | Bone | | |
|---|---|---|---|
| | destruction progression | | |
| | Yes | No | |
| Patient characteristics | (n = 24) | (n = 184) | P value † |
| Age (years) | 50 (41-62) | 54 (46-62) | 0.422 |
| Disease duration (years) | 2 (1-4) | 5 (2-12) | 0.001 |
| Female, n (%) | 20 (83.3) | 156 (83.4) | 0.999 ∫ |
| Current smoker, n (%) | 3 (12.5) | 23 (12.5) | 0.999 ∫ |
| Blood pressure, mmHg | | | |
| Systolic | 120.2 ± 11.5 | 120.3 ± 11.5 | 0.548 |
| Diastolic | 80.3 ± 7.3 | 76.9 ± 9.6 | 0.794 |
| Diabetes, n (%) | 2 (8.3) | 22 (13.4) | 0.552 ∫ |
| Body mass index, kg/m² | 22.2 ± 3.5 | 22.6 ± 3.3 | 0.529 |
| Glomerular filtration rate, | 112 (89-129) | 95 (80-112) | 0.016 |
| ml/min/1.73 m² | | | |
| Creatinine, mg/dl | 0.7 (0.6-0.8) | 0.6 (0.6-0.8) | 0.02 |
| Erythrocyte sedimentation | 66 (46-91) | 29 (16-51) | <0.001 |
| rate, mm/hr | | | |
| C-reactive protein, mg/dl | 3.3 (1.6-4.7) | 0.4 (0.1-1.3) | <0.001 |
| Rheumatoid factor, n (%) II | 20 (83.3) | 125 (67.9) | 0.123 ∫ |
| ACPA, n (%) II | 23 (95.8) | 156 (84.3) | 0.211 ∫ |
| Disease activity status at | 4.7 (4.2-6.2) | 4.0 (3.0-5.1) | 0.085 |
| 28 joints | | | |
| Prednisolone, n (%) | 19 (79.2) | 143 (77.7) | 0.807 ∫ |
| Methotrexate, n (%) | 14 (58.3) | 119 (64.7) | 0.656 ∫ |
| Hydroxychloroquine, n (%) | 19 (79.2) | 127 (69.0) | 0.349 ∫ |
| Sulfasalazine, n (%) | 8 (33.3) | 101 (54.9) | 0.081 ∫ |
| Leflunomide, n (%) | 11 (45.8) | 108 (58.7) | 0.283 ∫ |
| Anti-TNF-alpha, n (%) | 3 (12.5) | 13 (7.1) | 0.402 ∫ |
| Statin, n (%) | 2 (8.3) | 21 (11.4) | 0.752 ∫ |
| Non-steroid anti-inflammatory | 14 (58.3) | 121 (65.8) | 0.652 ∫ |
| agent, n (%) | | | |

* Data represent average ± standard deviation, median (interquartile range), or number (percent).
† Unless otherwise noted, Mann-Whitney U test was used.
∫ Fisher's two-tailed exact test was used.
II represents antibody positivity.

Then, a multivariate analysis, which corrected the known risk factors and all the factors that were significant in this patient group, was performed, and it was confirmed that increase in the urine AGP2 level increased the risk of bone destruction by 1.245 times (95% Confidence Interval: CI [1.006-1.541], P=0.044) three years later (Table 3).

TABLE 3
Multivariate logistic regression analysis results of urine protein
markers for bone destruction prediction

| | Odds ratio | |
|---|---|---|
| | (95% confidence | |
| | interval) | P value |
| Model 1 | | |
| (Baseline age, disease duration, | | |
| rheumatoid factor positivity, | | |
| anti-cyclic citrullinated peptide | | |
| antibody positivity, glomerular | | |
| filtration rate, C-reactive protein) | | |
| Model 1 + AGP2 | 1.245 (1.006-1.541) | 0.044 |

Model 1 includes the following basic variables: baseline age, disease duration, rheumatoid factor positivity, anti-cyclic citrullinated peptide antibody positivity, glomerular filtration rate and CRP.
To find Model 1-corrected odds ratio in the group that bone destruction progressed, the logistic regression analysis was used.

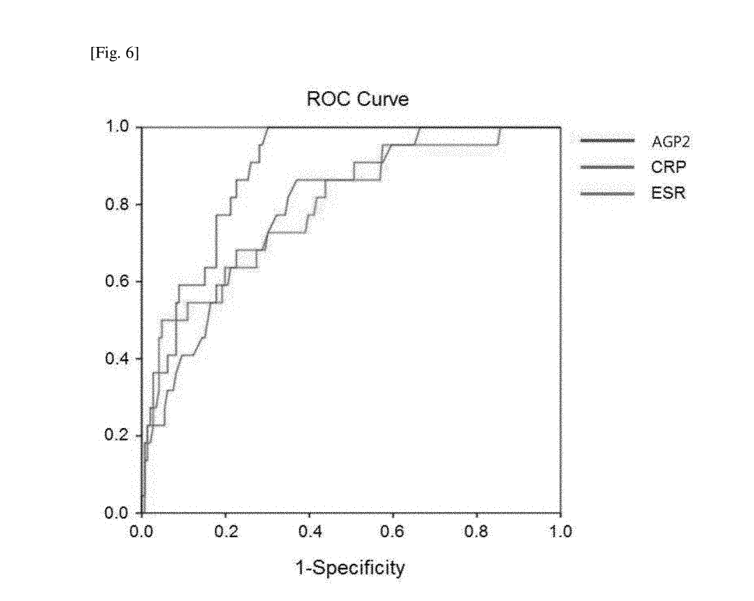

Next, to compare the diagnostic power with CRP, which is an existing well-known risk factor for predicting bone destruction, an ROC (receiver operating characteristic) curve analysis was performed. As a result of analysis, the AUC (area under the ROC curve) of AGP2 was 0.792 [0.688-0.895], which was almost similar to the AUC 0.887 [0.834-0.941] of CRP (FIG. 6).

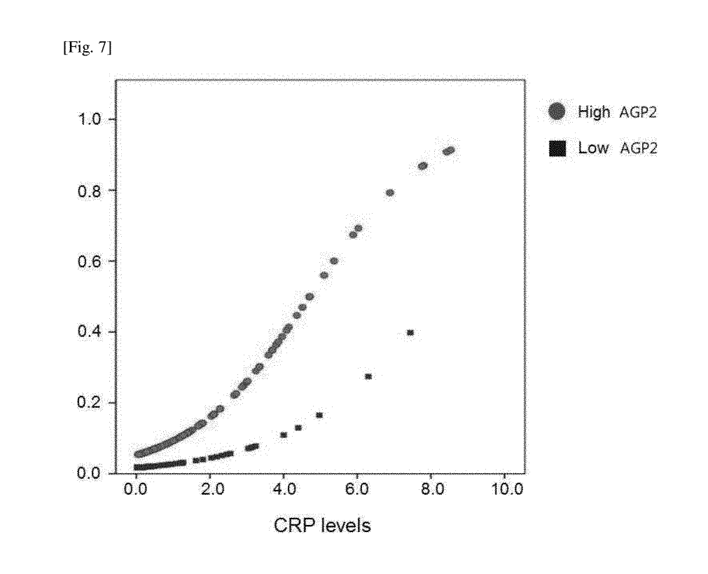

In addition, it was investigated using a probability plot whether the possibility of prediction of bone destruction is improved when the urine AGP2 is used in combination with blood CRP. When the case, in which the urinary AGP2 level is 2.210 ng/ml or more, was defined as high AGP2 group and the case less than the value was defined as low AGP2 group, the probability of predicting bone destruction three years later was increased, as CRP was increased, and it could be confirmed that this prediction was significantly increased in the high AGP2 group compared to the low AGP2 group (FIG. 7).

Considering that CRP and AGP2 can complement each other to predict bone destruction based on these results, each patient group was further divided into a positive group and a negative group, respectively and the risk was calculated by CRP-AGP2, ESR-AGP2, and ESR-CRP combinations. As a result, it was investigated that a group which is CRP-positive and AGP2-positive in the CRP-AGP2 combination increases the risk of bone destruction by 46.45 times compared to a group that both are negative (Table 4). Such a research result may suggest that the use of biomarkers in urine, which is a completely new approach, is nearly equal to disease activity or disease severity assessment using blood markers to date, and when used in combination, the complementary accuracy can be greatly increased.

TABLE 4
Multivariate logistic regression analysis results of combinations of
serum markers and urine protein markers for bone destruction prediction

| | Odds ratio | |
|---|---|---|
| | (95% confidence interval) | P value |
| Model 1 + AGP2-CRP combination | | |
| negative | 1 | |
| one positive | 8.715 (1.012-77.681) | 0.048 |
| two positive | 46.454 (5.228-412.732) | 0.001 |
| Model 1 + AGP2-ESR combination | | |
| Negative | 1 | |
| one positive | 1.091 (0.246-4.837) | 0.309 |
| two positive | 3.938 (1.101-15.686) | 0.042 |
| Model 1 + CRP-ESR combination | | |
| Negative | 1 | |
| one positive | 1.698 (0.347-3.844) | 0.268 |
| two positive | 4.086 (1.074-15.548) | 0.039 |

AGP represents alpha-1-acid glycoprotein, CRP represents C-reactive protein, and ESR represents erythrocyte sedimentation rate.
Model 1 includes the following basic variables: disease duration, rheumatoid factor positivity, anti-cyclic citrullinated peptide (CCP) antibody positivity and glomerular filtration rate.
To find Model 1-corrected odds ratio in the group that bone destruction progressed, the logistic regression analysis was used.

1.-4. (canceled)

5. A method for detecting cathepsin A from a urine sample of a patient to provide information necessary for diagnosis of rheumatoid arthritis.

6. The method according to claim 5, wherein said detection of cathepsin A is carried out using an antibody specific to cathepsin A.

7. The method according to claim 5, wherein said detection of cathepsin A is carried out using Western blot, an ELISA (enzyme linked immunosorbent assay), an immunoprecipitation assay, a complement fixation assay, a flow cytometry (Fluorescence Activated Cell Sorter, FACS), or a protein chip.

8.-11. (canceled)

12. A method for detecting cathepsin A of a rheumatoid arthritis patient to provide information necessary for diagnosis of osteoporosis in the rheumatoid arthritis patient.

13. The method according to claim 12, wherein said detection of cathepsin A is performed using an antibody specific to cathepsin A.

14. The method according to claim 12, wherein said detection of cathepsin A is performed using Western blot, an ELISA (enzyme linked immunosorbent assay), an immunoprecipitation assay, a complement fixation assay, a flow cytometry (Fluorescence Activated Cell Sorter, FACS), or a protein chip.

15.-23. (canceled)
